(19) United States
(12) Patent Application Publication
(10) Pub. No.: US 2025/0073290 A1
(43) Pub. Date: Mar. 6, 2025

(54) METHOD FOR INCREASING CALCIUM ABSORPTION USING CULTURE OF LACTOBACILLUS PLANTARUM PL-02

(71) Applicant: GLAC BIOTECH CO., LTD., Tainan City (TW)

(72) Inventors: Hsieh-Hsun HO, Tainan City (TW); Jui-Fen CHEN, Tainan City (TW); Yi-Wei KUO, Tainan City (TW); Chi-Huei LIN, Tainan City (TW); Ko-Chiang HSIA, Tainan City (TW); Shin-Yu TSAI, Tainan City (TW)

(21) Appl. No.: 18/806,076

(22) Filed: Aug. 15, 2024

(30) Foreign Application Priority Data
Aug. 31, 2023 (CN) 202311113005.0

(51) Int. Cl. A61K 35/747; A61K 33/06; A61K 35/745; A61P 3/14

(52) U.S. Cl. CPC A61K 35/747; A61K 33/06; A61K 35/745; A61P 3/14

(57) ABSTRACT

A method for increasing calcium absorption includes administering to a subject in need thereof a composition which contains a culture of Lactobacillus plantarum PL-02. The Lactobacillus plantarum PL-02 is deposited at the China General Microbiological Culture Collection Center (CGMCC) under an accession number CGMCC 20485 in accordance with the Budapest Treaty.

### CROSS-REFERENCE TO RELATED APPLICATION

This application claims priority to Chinese Invention Patent application Ser. No. 20/231,1113005.0 filed on Aug. 31, 2023, the entire disclosure of which is incorporated by reference herein.

### SEQUENCE LISTING XML

The Sequence Listing submitted concurrently herewith with a file name of “PE-70170-AM-SEQUENCE LISTING.xml,” a creation date of Jul. 2, 2024, and a size of 5.12 kilobytes, is part of the specification and is incorporated by reference in its entirety.

### FIELD

The present disclosure relates to a method for increasing calcium absorption using a composition that includes a culture of Lactobacillus plantarum PL-02.

### BACKGROUND

Calcium is an essential element required in large amounts by a human body, and plays an important role in blood coagulation, muscle contraction, bone construction, and maintenance of normal heart rhythm and nerve function in the human body. However, a lack of calcium may lead to various calcium deficiency-associated disorders.

Prevalent ways to increase calcium intake for delaying or alleviating a calcium deficiency-associated disorder include increase of exercise and use of calcium supplements, such as calcium carbonate, calcium oxide, calcium phosphate, and calcium lactate gluconate. Nevertheless, calcium contained in the calcium supplements may not be absorbed effectively by the intestine of a human body, while excessive use of the calcium supplements may increase urinary calcium level in the human body, resulting in formation of calcium-containing kidney stones.

Probiotics are resident normal flora of an intestinal tract and are believed to play important roles in regulating proper intestinal immunity and digestion by balancing intestinal microflora. These beneficial microorganisms are widely used as live microbial dietary supplements and can help with restoring intestinal microflora balance. Many species of lactic acid bacteria (LAB), such as Lactobacillus spp., Lactococcus spp., Pediococcus spp., Streptococcus spp., Enterococcus spp., Bifidobacterium spp., Bacillus spp., and Leuconostoc spp., are conferred with the generally recognized as safe (GRAS) status, and are widely used as probiotics.

It has been reported, in Huang W. C. et al., (2019), Nutrients, 19; 11 (11): 2836, doi: 10.3390/nu11112836., that administration of Lactobacillus plantarum TWK10 could effectively elevate the exercise performance in a dose-dependent manner, improve the fatigue-associated features correlated with better physiological adaptation, decrease body fat and increase muscle mass in healthy subjects.

In addition, it has been reported, in Hsu H. Y. et al., (2022), Microorganisms, 7; 10 (4): 784, doi: 10.3390/microorganisms10040784., that Lactobacillus plantarum TWK10 had high potential of being safe for consumption as a probiotic and administration of Lactobacillus plantarum TWK10 had no effect on hematological and biochemical parameters, including the calcium content, in the serum of Sprague-Dawley (SD) rats.

In view of the aforesaid, there is still a need to develop an effective way for increasing calcium absorption.

### SUMMARY

Therefore, an object of the present disclosure is to provide a method for increasing calcium absorption, which can alleviate at least one of the drawbacks of the prior art, and which includes administering to a subject in need thereof a composition including a culture of Lactobacillus plantarum PL-02.

The Lactobacillus plantarum PL-02 is deposited at the China General Microbiological Culture Collection Center (CGMCC) under an accession number CGMCC 20485 in accordance with the Budapest Treaty.

### BRIEF DESCRIPTION OF THE DRAWINGS

Other features and advantages of the disclosure will become apparent in the following detailed description of the embodiment(s) with reference to the accompanying drawings. It is noted that various features may not be drawn to scale.

FIG. 1 shows the relative fold change in calcium ion transport determined in the Ca control group, experimental groups SS and MS3, and comparative groups SS1 to SS4 and MS1 to MS5 of Example 1, infra, in which the symbols “*” and “*” represent p<0.05 and p<0.01, respectively (compared with the Ca control group).

FIG. 2 shows the relative fold change in calcium ion transport determined in the Ca control group, comparative group MS1, and experimental groups MS1 to MS5 of Example 1, infra, in which the symbol “**” represents p<0.01 (compared with the Ca control group).

FIG. 3 shows the relative fold change in calcium ion transport determined in the Ca control group, experimental group SB, and comparative groups SB1 to SB4 of Example 1, infra, in which the symbols “*” and “***” represent p<0.05 and p<0.001, respectively (compared with the Ca control group).

FIG. 4 shows the relative fold change in TRPV6 gene expression determined in the Ca control group, experimental group, and comparative groups 1 to 4 of Example 2, infra, in which the symbol “*” represents p<0.05 (compared with the Ca control group).

### DETAILED DESCRIPTION

For the purpose of this specification, it will be clearly understood that the word “comprising” means “including but not limited to”, and that the word “comprises” has a corresponding meaning.

It is to be understood that, if any prior art publication is referred to herein, such reference does not constitute an admission that the publication forms a part of the common general knowledge in the art, in Taiwan or any other country.

Unless defined otherwise, all technical and scientific terms used herein have the meaning commonly understood by a person skilled in the art to which the present disclosure belongs. One skilled in the art will recognize many methods and materials similar or equivalent to those described herein, which could be used in the practice of the present disclosure. Indeed, the present disclosure is in no way limited to the methods and materials described.

The present disclosure provides a method for increasing calcium absorption, which includes administering to a subject in need thereof a composition including a culture of Lactobacillus plantarum PL-02.

The Lactobacillus plantarum PL-02 is deposited at the China General Microbiological Culture Collection Center (CGMCC) under an accession number CGMCC 20485 in accordance with the Budapest Treaty.

As used herein, the term “administration” can be used interchangeably with other term such as “administering”, and means introducing, providing or delivering a pre-determined active ingredient to a subject by any suitable routes to perform its intended function.

As used herein, the term “subject” refers to any animal of interest, such as humans, monkeys, cows, sheep, horses, pigs, goats, dogs, cats, mice, and rats. In certain embodiments, the subject is a human.

According to the present disclosure, the subject suffers from a calcium deficiency-associated disorder. In certain embodiments, the calcium deficiency-associated disorder may be selected from the group consisting of osteopenia, rickets, joint pain, muscle cramp, hypertension, palpitation, anxiety, obesity, skin inflammation, hypocalcemia, and combinations thereof.

In certain embodiments, the subject may suffer from dysmenorrhea, or be in a perimenopausal, menopausal, or postmenopausal stage, resulting in a calcium deficiency.

In certain embodiments, the subject does not suffer from osteoporosis.

According to the present disclosure, the culture of the lactic acid bacterial strain (i.e., the Lactobacillus plantarum PL-02) may be prepared by culturing the abovementioned lactic acid bacterial strain in a liquid or solid medium suitable for growth and/or proliferation thereof.

As used herein, the term “culturing” can be used interchangeably with other terms such as “fermentation” and “cultivation”.

According to the present disclosure, the liquid medium suitable for use in this disclosure may be formulated using techniques well-known to those skilled in the art, or may be obtained as a commercial product which may include, but is not limited to, MRS (De Man, Rogosa and Sharpe) broth, MRS broth containing cysteine, and mineral medium containing glucose and soy peptone.

The procedures and conditions for cultivation may be adjusted according to practical requirements. In this regard, those skilled in the art may refer to journal articles, e.g., Hsieh P. S. et al. (2013), New Microbiol., 36:167-179.

According to the present disclosure, cultivation may be conducted at a temperature ranging from 25° C. to 40° C. for a time period ranging from 20 hours to 40 hours. In an exemplary embodiment, cultivation is conducted at 37° C. for 24 hours.

According to the present disclosure, the culture of the lactic acid bacterial strain is a liquid culture.

According to the present disclosure, the liquid culture may have a total bacterial concentration ranging from 10⁶ CFU/mL to 10¹² CFU/mL. In certain embodiments, the liquid culture may have a total bacterial concentration ranging from 10⁶ CFU/mL to 10¹⁰ CFU/mL. In an exemplary embodiment, the liquid culture may have a total bacterial concentration of 9×10⁶ CFU/mL. In another exemplary embodiment, the liquid culture may have a total bacterial concentration of 9×10⁷ CFU/mL.

According to the present disclosure, the liquid culture may be free of bacterial cells, which is obtained by subjecting a culture formed after culturing the lactic acid bacterial strain to a solid-liquid separation treatment.

According to the present disclosure, the solid-liquid separation treatment may be performed using techniques well-known to those skilled in the art. Examples of the solid-liquid separation treatment may include, but are not limited to, a centrifugation treatment, a filtration treatment, a concentration treatment, and combinations thereof. In an exemplary embodiment, the liquid culture is subjected to a centrifugation treatment to remove the bacterial cells thereof, so as to obtain a cell culture supernatant.

According to the present disclosure, the liquid culture may be subjected to a sterilization treatment, and hence may be free of viable bacterial cells.

According to the present disclosure, the liquid culture may contain viable bacterial cells, which is obtained by subjecting a culture formed after culturing the lactic acid bacterial strain to the aforesaid solid-liquid separation treatment so as to remove a liquid portion therefrom and then adding fresh medium thereto.

According to the present disclosure, the culture may be concentrated or non-concentrated, a liquid, a paste, a semi-solid, or a solid (e.g., a pellet, a granule, or a powder), and may be heat-inactivated, frozen, dried, or freeze-dried (e.g., may be in freeze-dried form or spray/fluid bed dried form).

According to the present disclosure, the composition may further include a culture of a probiotic microorganism selected from the group consisting of Lactococcus lactis LY-66 (BCRC 911055, CGMCC 21838), Bifidobacterium longum subsp. longum OLP-01 (BCRC 910875, CGMCC 17345), and a combination thereof. In certain embodiments, the composition includes liquid cultures of Lactobacillus plantarum PL-02, Lactococcus lactis LY-66, and Bifidobacterium longum subsp. longum OLP-01.

In certain embodiments, when the liquid cultures of Lactobacillus plantarum PL-02, Lactococcus lactis LY-66, and Bifidobacterium longum subsp. longum OLP-01 are cell culture supernatants, the composition may include, based on the total volume of the composition, 10 vol % to 90 vol % of the cell culture supernatant of Lactobacillus plantarum PL-02, 5 vol % to 45 vol % of the cell culture supernatant of Lactococcus lactis LY-66, and 5 vol % to 45 vol % of the cell culture supernatant of Bifidobacterium longum subsp. longum OLP-01. In some exemplary embodiments, the composition may include, based on the total volume of the composition, 50 vol % to 90 vol % of the cell culture supernatant of Lactobacillus plantarum PL-02, 5 vol % to 25 vol % of the cell culture supernatant of Lactococcus lactis LY-66, and 5 vol % to 25 vol % of the cell culture supernatant of Bifidobacterium longum subsp. longum OLP-01.

In certain embodiments, when the liquid cultures of Lactobacillus plantarum PL-02, Lactococcus lactis LY-66, and Bifidobacterium longum subsp. longum OLP-01 are the cell culture supernatants, a volume ratio of the cell culture supernatant of Lactobacillus plantarum PL-02, Lactococcus lactis LY-66, and Bifidobacterium longum subsp. longum OLP-01 in the composition ranges from 1:0.2:0.2 to 1:5:5. In an exemplary embodiment, the volume ratio of the cell culture supernatants of Lactobacillus plantarum PL-02, Lactococcus lactis LY-66, and Bifidobacterium longum subsp. longum OLP-01 in the composition is 1:0.5:0.5.

In certain embodiments, when the liquid cultures of Lactobacillus plantarum PL-02, Lactococcus lactis LY-66, and Bifidobacterium longum subsp. longum OLP-01 contain viable bacterial cells, the composition may include, based on the total volume of the composition, 10 vol % to 90 vol % of the liquid culture of Lactobacillus plantarum PL-02, 5 vol % to 45 vol % of the liquid culture of Lactococcus lactis LY-66, and 5 vol % to 45 vol % of the liquid culture of Bifidobacterium longum subsp. longum OLP-01. In some exemplary embodiments, the composition may include, based on the total volume of the composition, 50 vol % to 90 vol % of the liquid culture of Lactobacillus plantarum PL-02, 5 vol % to 25 vol % of the liquid culture of Lactococcus lactis LY-66, and 5 vol % to 25 vol % of the liquid culture of Bifidobacterium longum subsp. longum OLP-01.

In certain embodiments, when the liquid cultures of Lactobacillus plantarum PL-02, Lactococcus lactis LY-66, and Bifidobacterium longum subsp. longum OLP-01 contain viable bacterial cells, a volume ratio of the liquid cultures of Lactobacillus plantarum PL-02, Lactococcus lactis LY-66, and Bifidobacterium longum subsp. longum OLP-01 in the composition ranges from 1:0.2:0.2 to 1:5:5. In an exemplary embodiment, the volume ratio of the liquid cultures of Lactobacillus plantarum PL-02, Lactococcus lactis LY-66, and Bifidobacterium longum subsp. longum OLP-01 in the composition is 1:0.5:0.5.

In certain embodiments, when the liquid cultures of Lactobacillus plantarum PL-02, Lactococcus lactis LY-66, and Bifidobacterium longum subsp. longum OLP-01 contain viable bacterial cells, a number ratio (unit of bacteria number: CFU/mL) of Lactobacillus plantarum PL-02, Lactococcus lactis LY-66, and Bifidobacterium longum subsp. longum OLP-01 in the composition ranges from 1:0.2:0.2 to 1:5:5. In an exemplary embodiment, the number ratio (unit of bacteria number: CFU/mL) of Lactobacillus plantarum PL-02, Lactococcus lactis LY-66, and Bifidobacterium longum subsp. longum OLP-01 in the composition is 1:0.5:0.5.

According to the present disclosure, the composition may further include a calcium supplement. Examples of the calcium supplement may include, but are not limited to, calcium chloride, calcium carbonate, calcium oxide, calcium dihydrogen phosphate, dicalcium phosphate, tricalcium bis(phosphate), calcium lactate gluconate, calcium L-threonate, calcium citrate, calcium lactate, and combinations thereof. In an exemplary embodiment, the calcium supplement is calcium chloride.

According to the present disclosure, the composition may be formulated as a food product using a standard technique well known to one of ordinary skill in the art. For example, the composition may be formulated in the form of a food additive, which is added to an edible material to prepare a food product for human or animal consumption.

As used herein, the term “food product” refers to any article or substance that can be ingested by a subject into the body thereof. Examples of the food product may include, but are not limited to, milk powders, fermented milk, yogurt, butter, beverages (e.g., tea, coffee, etc.), functional beverages, a flour product, baked foods, confectionery, candies, fermented foods, animal feeds, health foods, infant foods, and dietary supplements.

According to the present disclosure, the composition may be prepared in the form of a pharmaceutical composition. The pharmaceutical composition may be formulated into a suitable dosage form for oral, topical or parenteral administration using technology well known to those skilled in the art.

According to the present disclosure, examples of the dosage form suitable for oral administration may include, but are not limited to, sterile powders, tablets, troches, lozenges, pellets, capsules, dispersible powders or granules, solutions, suspensions, emulsions, syrup, elixir, slurry, and the like.

According to the present disclosure, the pharmaceutical composition may be formulated into an external preparation suitable for topical application to the skin using technology well known to those skilled in the art. Examples of the external preparation may include, but are not limited to, emulsions, gels, ointments, creams, patches, liniments, powders, aerosols, sprays, lotions, serums, pastes, foams, drops, suspensions, salves, and bandages.

The parenteral administration, the pharmaceutical composition according to the present disclosure may be formulated into an injection, e.g., a sterile aqueous solution or a dispersion.

The pharmaceutical composition according to the present disclosure may be administered via one of the following parenteral routes: intraperitoneal injection, intrapleural injection, intramuscular injection, intravenous injection, intraarterial injection, intraarticular injection, intrasynovial injection, intraepidermal injection, subcutaneous injection, intradermal injection, intralesional injection, and sublingual administration.

According to the present disclosure, the pharmaceutical composition may further include a pharmaceutically acceptable carrier widely employed in the art of drug-manufacturing. For instance, the pharmaceutically acceptable carrier may include one or more of the following agents: solvents, buffers, emulsifiers, suspending agents, decomposers, disintegrating agents, dispersing agents, binding agents, excipients, stabilizing agents, chelating agents, diluents, gelling agents, preservatives, wetting agents, lubricants, absorption delaying agents, liposomes, and the like. The choice and amount of the aforesaid agents are within the expertise and routine skills of those skilled in the art.

According to the present disclosure, the dose and frequency of administration of the probiotic culture may vary depending on the following factors: the severity of the illness or disorder to be treated, routes of administration, and weight, age, physical condition and response of the subject to be treated. In general, the probiotic culture may be administered in a single dose or in several doses.

The disclosure will be further described by way of the following examples. However, it should be understood that the following examples are solely intended for the purpose of illustration and should not be construed as limiting the disclosure in practice.

### EXAMPLES

### General Experimental Materials

### 1. Source and Cultivation of Human Colon Adenocarcinoma Cell Line Caco-2

Human colon adenocarcinoma cell line Caco-2 (ATCC® HTB-37™) was purchased from the American Type Culture Collection (ATCC, Manassas, Va., USA). The Caco-2 cells were grown in a 10-cm Petri dish containing Dulbecco's Modified Eagle's Medium (DMEM, Manufacturer: Cytiva) supplemented with 10% fetal bovine serum (FBS, Manufacturer: Gibco, Cat. no.: 26140079) and 1% penicillin-streptomycin (Manufacturer: Cytiva). The Caco-2 cells were cultivated in an incubator at 37° C. with 5% CO₂. Medium change was performed every two to three days. Cell passage was performed when the cultured cells reached 80% to 90% of confluence.

### 2. Lactic Acid Bacterial (LAB) Strains

Lactobacillus plantarum PL-02 (which is disclosed in TW 1764356 B), Lactococcus lactis LY-66 (which is disclosed in TW 1782550 B), and Bifidobacterium longum subsp. longum OLP-01 (which is disclosed in TW 1764356 B) have been deposited at the Bioresource Collection and Research Center (BCRC) of the Food Industry Research and Development Institute (FIRDI) (No. 331, Shih-Pin Rd., Hsinchu City 300, Taiwan), and are known and readily available to the public. In addition, these LAB strains have also been deposited under the terms of the Budapest Treaty on the International Recognition of the Deposit of Microorganisms for the Purpose of Patent Procedure at the International Depositary Authority, i.e., China General Microbiological Culture Collection Center (CGMCC) of Chinese Academy of Sciences, the Institute of Microbiology (No. 1, West Beichen Rd., Chaoyang District, Beijing 100101, China) in accordance with the Budapest Treaty.

The relevant information regarding each of the LAB strains (including accession number and date of deposit) is listed in Table 1 below.

TABLE 1

| LAB strains | Accession number | Date of deposit |
|---|---|---|
| Lactobacillus plantarum | BCRC 911012 | Jul. 28, 2020 |
| PL-02 | CGMCC 20485 | Aug. 6, 2020 |
| Bifidobacterium longum | BCRC 910875 | Feb. 22, 2019 |
| subsp. longum OLP-01 | CGMCC 17345 | Mar. 18, 2019 |
| Lactococcus lactis LY-66 | BCRC 911055 | Apr. 23, 2021 |
| | CGMCC 21838 | Feb. 26, 2021 |

In addition, the Lactobacillus plantarum strains used as comparative strains include Lactobacillus plantarum 299v purchased from Japan Ace International Merchandise Co., Ltd., Lactobacillus plantarum TWK10 purchased from GenMont Biotech Inc., Lactobacillus plantarum GMNL-662 purchased from Synbio Tech Inc., and Lactobacillus plantarum gL-299 isolated by the applicant from commercially available pickled cabbage.

### 3. Preparation of Bacterial Suspensions and Cell Culture Supernatants of LAB Strains Containing 5 mM Calcium Chloride

A respective one of the seven LAB strains described in section 2 of “General Experimental Materials” was inoculated in a MRS broth (Manufacturer: Difco, Cat. no. 288130), followed by cultivation in an incubator (37° C., 5% CO₂) under a facultative anaerobic condition for 24 hours, so as to obtain a respective inoculum. Thereafter, the respective inoculum was inoculated in an amount of 2% (v/v) into a MRS broth, followed by cultivation in an incubator (37° C., 5% CO₂) under a facultative anaerobic condition for 24 hours. After centrifugation at 3,000 rpm and 4° C. for 10 minutes, the resultant cell culture supernatant and cell pellet were collected. Then, an appropriate amount of calcium chloride (Manufacturer: Sigma-Aldrich, Cat. no.: V900269) was added to the cell culture supernatant, so as to obtain a cell culture supernatant containing 5 mM calcium chloride (abbreviated as CaCl₂-cell culture supernatant).

In addition, with regard to the cell pellet for the five Lactobacillus plantarum strains, an appropriate amount of DMEM supplemented with 5 mM calcium chloride was added to suspend the cell pellet and to adjust the bacterial concentration to the desired bacterial concentration which was determined using a plate counting medium, thereby obtaining a bacterial suspension containing 5 mM calcium chloride and having a bacterial concentration of 9×10⁶ CFU/mL or 9×10⁷ CFU/mL (abbreviated as CaCl₂-bacterial suspension).

The resultant CaCl₂-cell culture supernatants and CaCl₂-bacterial suspensions were used in the following experiments.

### General Procedures

### 1. Statistical Analysis

All the experiments described below were performed in triplicates. The experimental data of all the groups are expressed as mean±standard deviation (SD), and were analyzed using Student's t-test, so as to evaluate the differences between the groups. Statistical significance is indicated by p<0.05.

### Example 1. Evaluation of the effect of Lactobacillus plantarum PL-02 on Improving Intestinal Calcium Ion Transport

### Experimental Procedures

First, the Caco-2 cells prepared in section 1 of “General Experimental Materials” were divided into 22 groups, including a blank control group, a Ca control group, seven experimental groups (i.e., experimental groups SS, MS1 to MS5, and SB), and thirteen comparative groups (i.e., comparative groups SS1 to SS4, MS1 to MS5, and SB1 to SB4). Each group of the Caco-2 cells was seeded at a concentration of 2×10⁵ cells/well into a respective permeable Transwell® insert (Manufacturer: Corning Inc.). Each of the Transwell® inserts had a polycarbonate membrane with a pore size of 0.4 μm and contained 2.5 ml of DMEM. Next, the Transwell® inserts were placed into 6-well plates containing 1.5 mL of DMEM in each well, followed by cultivation in an incubator (37° C., 5% CO₂) for 6 days, and medium change was performed every 3 days, so that the Caco-2 cells formed a cell monolayer on a bottom of each Transwell® insert. Afterwards, the culture medium in each Transwell® insert was removed, and the respective Transwell® insert was washed with phosphate-buffered saline (PBS) two times. The Caco-2 cell monolayer of each of the experimental groups SS and SB and the comparative groups SS1 to SS4 and SB1 to SB4 was added with 1.5 ml of the respective one of the CaCl₂-cell culture supernatants or CaCl₂-bacterial suspensions having a bacterial concentration of 9×10⁶ CFU/mL (prepared in section 3 of “General Experimental Materials”) as shown in Table 2 below. In addition, the Caco-2 cell monolayer of each of the blank control group and the Ca control group was added with 1.5 mL of the respective testing agent as shown in Table 2 below.

Moreover, as shown in Table 3 below, the CaCl₂-cell culture supernatants of Bifidobacterium longum subsp. longum OLP-01 and Lactococcus lactis LY-66 prepared in section 3 of “General Experimental Materials” was mixed in equal volume percentage concentration (i.e., 50 vol %), and 25 vol % of the CaCl₂-cell culture supernatant of Bifidobacterium longum subsp. longum OLP-01 and 25 vol % of the CaCl₂-cell culture supernatants of Lactococcus lactis LY-66 were mixed with 50 vol % of the CaCl₂-cell culture supernatant of Lactobacillus plantarum PL-02, Lactobacillus plantarum GMNL-662, Lactobacillus plantarum 299v, Lactobacillus plantarum TWK10, or Lactobacillus plantarum gL-299 prepared in section 3 of “General Experimental Materials”. In addition, the CaCl₂-cell culture supernatants of Lactobacillus plantarum PL-02, Bifidobacterium longum subsp. longum OLP-01, and Lactococcus lactis LY-66 were mixed in different volume percentage concentrations (vol %) as shown in Table 3 below, so as to evaluate the effect of changes in the amount of the CaCl₂-cell culture supernatants of Lactobacillus plantarum PL-02, Bifidobacterium longum subsp. longum OLP-01, and Lactococcus lactis LY-66 on improving intestinal calcium ion transport. The resultant 10 supernatant mixtures (i.e., supernatant mixtures 1 to 10) and the contents thereof are shown in Table 3 below. Next, the Caco-2 cell monolayer of each of the experimental groups MS1 to MS5 and the comparative groups MS1 to MS5 was added with 1.5 mL of the respective one of the supernatant mixtures as shown in Table 4 below.

TABLE 2

| Group | Testing agent |
|---|---|
| Blank control group | DMEM |
| Ca control group | DMEM containing 5 mM CaCl₂ |
| Experimental group SS | CaCl₂-cell culture supernatant of |
| | Lactobacillus plantarum PL-02 |
| Comparative group SS1 | CaCl₂-cell culture supernatant of |
| | Lactobacillus plantarum GMNL-662 |
| Comparative group SS2 | CaCl₂-cell culture supernatant of |
| | Lactobacillus plantarum 299v |
| Comparative group SS3 | CaCl₂-cell culture supernatant of |
| | Lactobacillus plantarum TWK10 |
| Comparative group SS4 | CaCl₂-cell culture supernatant of |
| | Lactobacillus plantarum gL-299 |
| Experimental group SB | CaCl₂-bacterial suspension of |
| | Lactobacillus plantarum PL-02 |
| Comparative group SB1 | CaCl₂-bacterial suspension of |
| | Lactobacillus plantarum GMNL-662 |
| Comparative group SB2 | CaCl₂-bacterial suspension of |
| | Lactobacillus plantarum 299v |
| Comparative group SB3 | CaCl₂-bacterial suspension of |
| | Lactobacillus plantarum TWK10 |
| Comparative group SB4 | CaCl₂-bacterial suspension of |
| | Lactobacillus plantarum gL-299 |

TABLE 3

| | | Volume |
|---|---|---|
| | | percentage |
| | | concen- |
| Supernatant | | tration |
| mixture | LAB strain applied | (vol %) |
| 1 | Lactobacillus plantarum PL-02, | 10:45:45 |
| | Bifidobacterium longum subsp. longum OLP-01, | |
| | and Lactococcus lactis LY-66 | |
| 2 | Lactobacillus plantarum PL-02, | 30:35:35 |
| | Bifidobacterium longum subsp. longum OLP-01, | |
| | and Lactococcus lactis LY-66 | |
| 3 | Lactobacillus plantarum PL-02, | 50:25:25 |
| | Bifidobacterium longum subsp. longum OLP-01, | |
| | and Lactococcus lactis LY-66 | |
| 4 | Lactobacillus plantarum PL-02, | 70:15:15 |
| | Bifidobacterium longum subsp. longum OLP-01, | |
| | and Lactococcus lactis LY-66 | |
| 5 | Lactobacillus plantarum PL-02, | 90:5:5 |
| | Bifidobacterium longum subsp. longum OLP-01, | |
| | and Lactococcus lactis LY-66 | |
| 6 | Bifidobacterium longum subsp. longum OLP-01 | 50:50 |
| | and Lactococcus lactis LY-66 | |
| 7 | Lactobacillus plantarum GMNL-662, | 50:25:25 |
| | Bifidobacterium longum subsp. longum OLP-01, | |
| | and Lactococcus lactis LY-66 | |
| 8 | Lactobacillus plantarum 299v, | 50:25:25 |
| | Bifidobacterium longum subsp. longum OLP-01, | |
| | and Lactococcus lactis LY-66 | |
| 9 | Lactobacillus plantarum TWK10, | 50:25:25 |
| | Bifidobacterium longum subsp. longum OLP-01, | |
| | and Lactococcus lactis LY-66 | |
| 10 | Lactobacillus plantarum gL-299, | 50:25:25 |
| | Bifidobacterium longum subsp. longum OLP-01, | |
| | and Lactococcus lactis LY-66 | |

TABLE 4

| Group | Testing agent |
|---|---|
| Experimental group MS1 | Supernatant mixture 1 |
| Experimental group MS2 | Supernatant mixture 2 |
| Experimental group MS3 | Supernatant mixture 3 |
| Experimental group MS4 | Supernatant mixture 4 |
| Experimental group MS5 | Supernatant mixture 5 |
| Comparative group MS1 | Supernatant mixture 6 |
| Comparative group MS2 | Supernatant mixture 7 |
| Comparative group MS3 | Supernatant mixture 8 |
| Comparative group MS4 | Supernatant mixture 9 |
| Comparative group MS5 | Supernatant mixture 10 |

Thereafter, each group was cultivated in an incubator (37° C., 5% CO₂) for 24 hours. On the 30^{th} minute and 24^{th} hour after treatment with the testing agent, the liquid in each well was collected, and was then subjected to determination of calcium content using a Calcium Colorimetric Assay Kit (Manufacturer: BioVision, Cat. no.: K380-250).

The percentage increase in calcium content for each group was calculated by substituting the detected calcium content on the 30^{th} minute and 24^{th} hour after treatment into the following Equation (1):

A=B/C (1)

where A=percentage increase in calcium content
B=calcium content detected in each group on the 24^{th} hour after the treatment
C=calcium content detected in each group on the 30^{th} minute after the treatment

The relative fold change in calcium ion transport for each group was calculated by substituting the thus obtained percentage increase in calcium content into the following Equation (2):

D=(E−F)/(G−F) (2)

where D=relative fold change in calcium ion transport
E=percentage increase in calcium content for each group
F=percentage increase in calcium content for the blank control group
G=percentage increase in calcium content for the Ca control group

The data thus obtained were analyzed according to the procedures as described in section 1 of “General Procedures”.

### Results

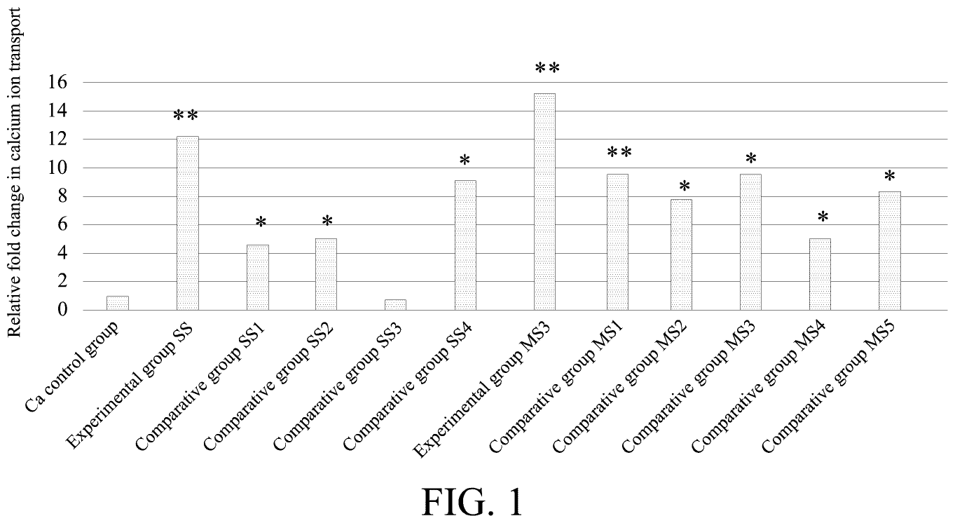

FIG. 1 shows the relative fold change in calcium ion transport determined in each of the Ca control group, the experimental groups SS and MS3, and the comparative groups SS1 to SS4 and MS1 to MS5 after treating the Caco-2 cell monolayer with the respective testing agent as shown in Table 2 and Table 4. As shown in FIG. 1, compared with the Ca control group, the relative fold change in calcium ion transport determined in each of the experimental group SS and the comparative groups SS1, SS2, and SS4 showed a significant increase. To be specific, the relative fold change in calcium ion transport determined in the experimental group SS was significantly higher than those in the comparative groups SS1, SS2, and SS4. However, compared with the Ca control group, the relative fold change in calcium ion transport determined in the comparative group SS3 showed a slight decrease. These results demonstrate that the cell culture supernatant of Lactobacillus plantarum PL-02 can effectively increase intestinal calcium ion transport, and such effect is significantly better than those of other Lactobacillus plantarum strains.

Moreover, compared with the comparative group MS1, the relative fold change in calcium ion transport determined in the experimental group MS3 showed an increase, while that in each of the comparative groups MS2 to MS5 showed no increase and even showed a decrease. These results indicate that the combination of Lactobacillus plantarum PL-02 with Bifidobacterium longum subsp. longum OLP-01 and Lactococcus lactis LY-66 can exhibit a synergistic effect in increasing the intestinal calcium ion transport.

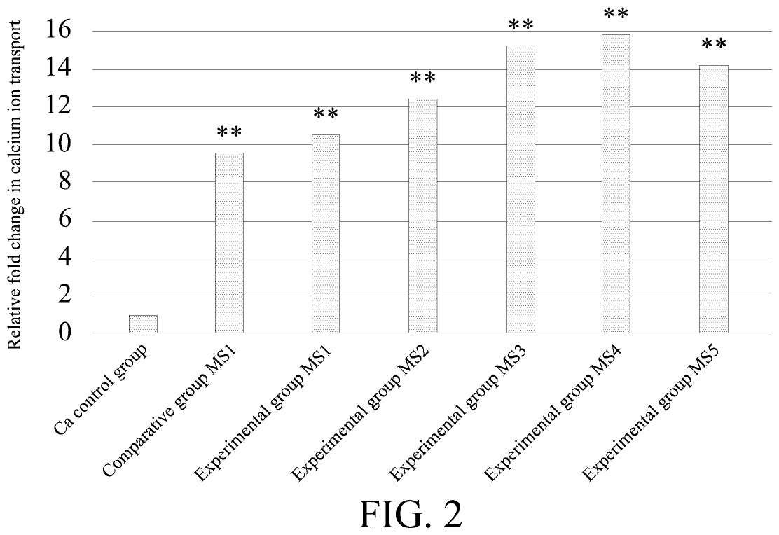

FIG. 2 shows the relative fold change in calcium ion transport determined in each of the Ca control group, the comparative group MS1, and the experimental groups MS1 to MS5 after treating the Caco-2 cell monolayer with the respective testing agent as shown in Table 2 and Table 4. As shown in FIG. 2, compared with the comparative group MS1, the relative fold change in calcium ion transport determined in each of the experimental groups MS1 to MS5 showed an increase. These results demonstrate that the cell culture supernatants of Lactobacillus plantarum PL-02, Bifidobacterium longum subsp. longum OLP-01, and Lactococcus lactis LY-66, when mixed in a specific volume percentage concentration (particularly from 10 vol % to 90 vol % of the cell culture supernatant of Lactobacillus plantarum PL-02) to prepare a supernatant mixture, can exhibit a synergistic effect in increasing the intestinal calcium ion transport.

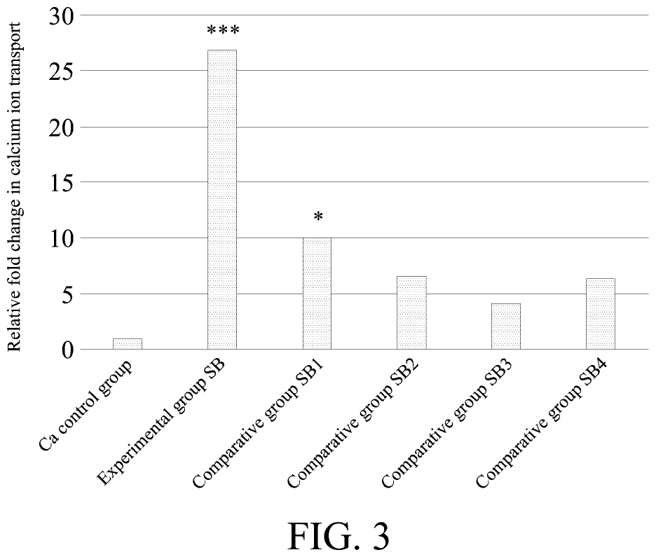

FIG. 3 shows the relative fold change in calcium ion transport determined in each of the Ca control group, the experimental group SB, and the comparative groups SB1 to SB4 after treating the Caco-2 cell monolayer with the respective testing agent as shown in Table 2. As shown in FIG. 3, compared with the Ca control group, the relative fold change in calcium ion transport determined in each of the comparative groups SB2 to SB4 showed a slight increase, while the relative fold change in calcium ion transport determined in each of the experimental group SB and the comparative group SB1 showed a significant increase. To be specific, the relative fold change in calcium ion transport determined in the experimental group SB was higher than that in each of the comparative groups SB1 to SB4 and was approximately 6.5 times higher than that in the comparative group SB3. These results indicate that the bacterial suspension of Lactobacillus plantarum PL-02 can effectively increase intestinal calcium ion transport, and such effect is significantly better than those of other Lactobacillus plantarum strains.

### Example 2. Evaluation of the Effect of Lactobacillus plantarum PL-02 on Increasing Expression of Transient Receptor Potential Vanilloid 6 (TRPV6) Calcium Channel Protein in Intestinal Cells

### Experimental Procedures

First, the Caco-2 cells prepared in section 1 of “General Experimental Materials” were divided into 7 groups, including a blank control group, a Ca control group, an experimental group, and four comparative groups (i.e., comparative groups 1 to 4). Each group of the Caco-2 cells was seeded at a concentration of 2×10⁵ cells/well into a respective well of 6-well culture plates containing 3 mL of DMEM supplemented with 10% FBS and 1% penicillin-streptomycin, followed by cultivation in an incubator (37° C., 5% CO₂) for 11 days. During the cultivation period, the culture medium in each group was removed to be replaced with a fresh culture medium every three days. At the end of the cultivation 5 period, the culture medium in each well was removed. The Caco-2 cells of each of the experimental group and the comparative groups 1 to 4 were added with 3 mL of the respective one of the CaCl₂-bacterial suspensions of different Lactobacillus plantarum strains having a bacterial concentration of 9×10⁷ CFU/mL (prepared in section 3 of “General Experimental Materials”) as shown in Table 5 below. In addition, the Caco-2 cells of each of the blank control group and the Ca control group were added with 3 mL of the respective testing agent as shown in Table 5 below, followed by cultivation in an incubator (37° C., 5% CO₂) for 6 hours.

TABLE 5

| Group | Testing agent |
|---|---|
| Blank control group | DMEM |
| Ca control group | DMEM containing 5 mM CaCl₂ |
| Experimental group | CaCl₂-bacterial suspension of Lactobacillus |
| | plantarum PL-02 |
| Comparative group 1 | CaCl₂-bacterial suspension of Lactobacillus |
| | plantarum GMNL-662 |
| Comparative group 2 | CaCl₂-bacterial suspension of Lactobacillus |
| | plantarum 299v |
| Comparative group 3 | CaCl₂-bacterial suspension of Lactobacillus |
| | plantarum TWK10 |
| Comparative group 4 | CaCl₂-bacterial suspension of Lactobacillus |
| | plantarum gL-299 |

Afterwards, the liquid in each well was removed and the cell culture of each group was subjected to total RNA extraction using a Total RNA Extraction Miniprep System (Manufacturer: Viogene, Cat. no.: GR1001) in accordance with the manufacturer's instructions. The resultant total RNA of each group was used as a template for synthesizing cDNA by reverse transcription polymerase chain reaction (RT-PCR) using GoScript™ Reverse Transcriptase (Manufacturer: Promega, Cat. no.: A5003) in accordance with the manufacturer's instructions.

The thus obtained cDNA, serving as a DNA template, was subjected to quantitative real-time polymerase chain reaction (quantitative real-time PCR) based on SYBR-Green I fluorescence, which was performed on a StepOnePlus™ real-time PCR system (Manufacturer: Applied Biosystems™) using a designed primer pair specific for TRPV6 gene shown in Table 6 below and under the reaction conditions shown in Table 7 below. Glyceraldehyde-3-phosphate dehydrogenase (GAPDH) gene was used as an endogenous control in the quantitative real-time PCR analysis to normalize the gene expression data (see Table 6).

TABLE 6

| | | | Size of |
|---|---|---|---|
| | | | PCR |
| | | Nucleotide sequence | product |
| Target gene | Primer | (5′→3′) | (bp) |
| TRPV6 gene | Forward primer | ggacaacaccctcttacagca | 224 |
| (GenBank accession | TRPV6-F | (SEQ ID NO: 1) | |
| no. AK291707.1) | Reverse primer | ccagcaccatgaaggcata | |
| | TRPV6-R | (SEQ ID NO: 2) | |
| GAPDH gene | Forward primer | gaaggtgaaggtcggagt | 225 |
| (NCBI accession | GAPDH-F | (SEQ ID NO: 3) | |
| no. NM_002046.2) | Reverse primer | gaagatggtgatggatttc | |
| | GAPDH-R | (SEQ ID NO: 4) | |

TABLE 7

| Reaction mix | Volume (μL) |
|---|---|
| cDNA (0.1 μg/μL) | 2 |
| Forward primer (10 μM) | 0.8 |
| Reverse primer (10 μM) | 0.8 |
| qPCRBIO SyGreen Mix | 10 |
| (Manufacturer: PCR Biosystems, | |
| Cat. no.: PB20.12-05) | |
| Deionized water | 6.4 |

Operating conditions: forty cycles of the following reactions: denaturation at 95° C. for 3 minutes, annealing at 95° C. for 5 seconds, and extension at 60° C. for 30 seconds.

The resultant PCR product was subjected to determination of fluorescence intensity, followed by calculating the cycle threshold (Cₜ) value of TRPV6 gene. Quantitative real-time PCR data were analyzed using the comparative Cₜ method. Briefly, the Cₜ value of TRPV6 gene in each group was normalized with that of GAPDH gene, and the relative fold change in TRPV6 gene expression for each group was further calculated using the following Equation (3):

H=(I−J)/(K−J) (3)

where H=relative fold change in TRPV6 gene expression
I=normalized Cₜ value of TRPV6 gene in each group
J=normalized Cₜ value of TRPV6 gene in the blank control group
K=normalized Cₜ value of TRPV6 gene in the Ca control group

The data thus obtained were analyzed according to the procedures as described in section 1 of “General Procedures”.

### Results

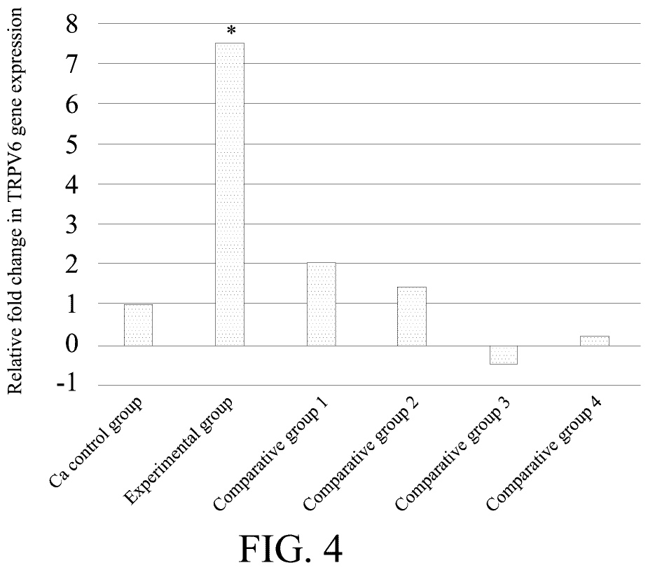

FIG. 4 shows the relative fold change in TRPV6 gene expression determined in each of the Ca control group, the experimental group, and the comparative groups 1 to 4 after treating the Caco-2 cells with the respective testing agent as shown in Table 5. As shown in FIG. 4, compared with the Ca control group, the relative fold change in TRPV6 gene expression determined in the experimental group showed a significant increase, and the relative fold change in TRPV6 gene expression determined in each of the comparative groups 1 and 2 showed a slight increase, while the relative fold change in TRPV6 gene expression determined in each of the comparative groups 3 and 4 showed a slight decrease. To be specific, the relative fold change in TRPV6 gene expression determined in the experimental group was more than three times higher than that of the comparative group 1. This result indicates that Lactobacillus plantarum PL-02 can effectively increase the TRPV6 expression in intestinal cells.

Summarizing the above test results, it is clear that compared with other Lactobacillus plantarum strains, the culture of Lactobacillus plantarum PL-02 (including the bacterial cells portion and the cell culture supernatant without bacterial cells) is capable of significantly increasing the intestinal calcium ion transport and the expression of the TRPV6 in intestinal cells, and hence can effectively increase calcium absorption.

In the description above, for the purposes of explanation, numerous specific details have been set forth in order to provide a thorough understanding of the embodiment(s). It will be apparent, however, to one skilled in the art, that one or more other embodiments may be practiced without some of these specific details. It should also be appreciated that reference throughout this specification to “one embodiment,” “an embodiment,” an embodiment with an indication of an ordinal number and so forth means that a particular feature, structure, or characteristic may be included in the practice of the disclosure. It should be further appreciated that in the description, various features are sometimes grouped together in a single embodiment, figure, or description thereof for the purpose of streamlining the disclosure and aiding in the understanding of various inventive aspects; such does not mean that every one of these features needs to be practiced with the presence of all the other features. In other words, in any described embodiment, when implementation of one or more features or specific details does not affect implementation of another one or more features or specific details, the one or more features may be singled out and practiced alone without the another one or more features or specific details. It should be further noted that one or more features or specific details from one embodiment may be practiced together with one or more features or specific details from another embodiment, where appropriate, in the practice of the disclosure.

While the disclosure has been described in connection with what is(are) considered the exemplary embodiment(s), it is understood that this disclosure is not limited to the disclosed embodiment(s) but is intended to cover various arrangements included within the spirit and scope of the broadest interpretation so as to encompass all such modifications and equivalent arrangements.

What is claimed is:

1. A method for increasing calcium absorption, comprising administering to a subject in need thereof a composition including a culture of Lactobacillus plantarum PL-02,
wherein the Lactobacillus plantarum PL-02 is deposited at the China General Microbiological Culture Collection Center (CGMCC) under an accession number CGMCC 20485 in accordance with the Budapest Treaty.

2. The method as claimed in claim 1, wherein the composition further includes a culture of a probiotic microorganism selected from the group consisting of Lactococcus lactis LY-66 which is deposited at the CGMCC under an accession number CGMCC 21838 in accordance with the Budapest Treaty, Bifidobacterium longum subsp. longum OLP-01 which is deposited at the CGMCC under an accession number CGMCC 17345 in accordance with the Budapest Treaty, and a combination thereof.

3. The method as claimed in claim 2, wherein the composition includes cultures of Lactobacillus plantarum PL-02, Lactococcus lactis LY-66, and Bifidobacterium longum subsp. longum OLP-01.

4. The method as claimed in claim 1, wherein the culture is a liquid culture.

5. The method as claimed in claim 4, wherein the liquid culture is free of bacterial cells.

6. The method as claimed in claim 4, wherein the liquid culture contains bacterial cells.

7. The method as claimed in claim 3, wherein the cultures of Lactobacillus plantarum PL-02, Lactococcus lactis LY-66, and Bifidobacterium longum subsp. longum OLP-01 are liquid cultures which are free of bacterial cells.

8. The method as claimed in claim 7, wherein a volume ratio of the liquid cultures of Lactobacillus plantarum PL-02, Lactococcus lactis LY-66, and Bifidobacterium longum subsp. longum OLP-01 in the composition ranges from 1:0.2:0.2 to 1:5:5.

9. The method as claimed in claim 3, wherein the cultures of Lactobacillus plantarum PL-02, Lactococcus lactis LY-66, and Bifidobacterium longum subsp. longum OLP-01 are liquid cultures which contain bacterial cells.

10. The method as claimed in claim 9, wherein a volume ratio of the liquid cultures of Lactobacillus plantarum PL-02, Lactococcus lactis LY-66, and Bifidobacterium longum subsp. longum OLP-01 in the composition ranges from 1:0.2:0.2 to 1:5:5.

11. The method as claimed in claim 9, wherein a number ratio of Lactobacillus plantarum PL-02, Lactococcus lactis LY-66, and Bifidobacterium longum subsp. longum OLP-01 in the composition ranges from 1:0.2:0.2 to 1:5:5.

12. The method as claimed in claim 1, wherein the subject suffers from a calcium deficiency-associated disorder.

13. The method as claimed in claim 1, wherein the composition further includes a calcium supplement.

14. The method as claimed in claim 1, wherein the composition is a food product or a pharmaceutical composition.

14. The method as claimed in claim 14, wherein the pharmaceutical composition is administered by a route selected from the group consisting of oral administration, topical administration, and parenteral administration.


SEQUENCE LISTING (4 sequences)

SEQ ID NO: 1 - DNA, 21 bp, synthetic construct
  source 1..21: mol_type = other DNA; note = forward primer for amplification of TRPV6
ggacaacaccctcttacagca

SEQ ID NO: 2 - DNA, 19 bp, synthetic construct
  source 1..19: mol_type = other DNA; note = reverse primer for amplification of TRPV6
ccagcaccatgaaggcata

SEQ ID NO: 3 - DNA, 18 bp, synthetic construct
  source 1..18: mol_type = other DNA; note = forward primer for amplification of GAPDH
gaaggtgaaggtcggagt

SEQ ID NO: 4 - DNA, 19 bp, synthetic construct
  source 1..19: mol_type = other DNA; note = reverse primer for amplification of GAPDH
gaagatggtgatggatttc